SEQUENCE LISTING (1 sequence)

SEQ ID NO: 1 - protein, 14 aa, Artificial
  Chemically synthesized
  MOD_RES (12)..(12): Xaa  is D-Arginine
FVPIFTYGELQXLK

(19) United States
(12) Patent Application Publication
(10) Pub. No.: US 2006/0287243 A1
(43) Pub. Date: Dec. 21, 2006

(54) Stable pharmaceutical compositions including motilin-like peptides

(76) Inventors: Navneet Puri, Bridgewater, NJ (US); Satish K. Pejaver, Bridgewater, NJ (US); Varun Sethi, Green Brook, NJ (US); Ralph A. Lessor, New Providence, NJ (US)

Correspondence Address: BAXTER HEALTHCARE CORPORATION, ONE BAXTER PARKWAY, DF2-2E, DEERFIELD, IL, 60015, US

(21) Appl. No.: 11/197,378

(22) Filed: Aug. 4, 2005

Related U.S. Application Data
(60) Provisional application No. 60/691,599, filed on Jun. 17, 2005.

(51) Int. Cl. A61K 38/10

(52) U.S. Cl. 514/15

(57) ABSTRACT

Stable, pharmaceutical compositions including a synthetic motilin-like peptide in a buffered aqueous solution or in an unbuffered aqueous solution are disclosed. The composition provides for a peptide that remains stable and substantially retains its initial potency during extended storage and after steam sterilization.

### CROSS-REFERENCE TO RELATED APPLICATIONS

This application claims the benefit of U.S. Provisional Application No. 60/691,599, filed Jun. 17, 2005, the contents of which is incorporated by reference.

The present invention relates to pharmaceutical compositions including a motilin-like peptide. More particularly, the present invention relates to pharmaceutical compositions including a motilin-like peptide that remains stable and retains its potency and binding affinity after extended periods of storage.

### BACKGROUND OF THE INVENTION

The peptide motilin is a gastrointestinal linear polypeptide hormone, which stimulates gastrointestinal motor activity in mammals. Motilin plays a role in increasing gastric motility by regulating the interdigestive myoelectric complex. Administration of motilin to human subjects accelerates intestinal transit and enhances gastric emptying [Christofides et al., Gastroenterology 76, 903-907 (1979)]. In vitro studies have shown that motilin stimulates contractions of human and rabbit duodenal smooth muscle strips and isolated gastrointestinal smooth muscle cells. Motilin has been reported to stimulate the emptying of solids and liquid in patients with diabetic gastroparesis. [Peeters et al., Gastroenterology 100, A480 (1991)], and has been used to treat patients with paralytic ileus caused by carcinoma of the gastrointestinal tract [Meyer et al., Med. Klin. 86, 515-517 (1991)].

One of the drawbacks of motilin is its relatively short half-life in vivo (Christofides, 1979, op. cit.], which makes it necessary to administer the hormone by continuous infusion to induce a therapeutic effect. In response to this shortcoming, synthetic motilin-like peptides, which mimic motilin activity while possessing enhanced metabolic stability, have been developed. These peptides are reported and described in U.S. Pat. No. 5,422,341, incorporated herein by reference.

The synthetic motilin-like peptides described in U.S. Pat. No. 5,422,341 have been shown to be effective in stimulating gastrointestinal activity, and demonstrate increased stability to biodegradation in relevant organ tissue homogenates. However, as with naturally occurring motilin, the synthetic motilin-like compounds are potentially subject to rapid degradation of the peptide due to hydrolysis, oxidation, or other chemical processes prior to administration.

It is known that certain peptides otherwise prone to degradation during storage can be stabilized and, thus, stored for extended periods without loss of biological activity, by preparing the peptide as an aqueous composition. For example, U.S. Pat. No. 5,482,931 discloses an aqueous composition for administration of peptides such as oxytocin, vasopressin and analogs and derivatives thereof, which can maintain stability over time at room temperature. The solution contains a buffer, a quarternary amine preservative or disinfectant and an osmotic-controlling agent. According to U.S. Pat. No. 5,482,931, the aqueous peptide composition retained useful shelf life after several weeks of storage at room temperature.

Other examples of stable aqueous peptide formulation are disclosed in U.S. Pat. Nos. 5,916,582 and 6,068,850. These patents describe aqueous peptide formulations of high-concentration lutenizing hormone-releasing hormone (LHRH), which may be stored at elevated temperatures (e.g., 37° C.) for long periods of time.

One example of a “stable” motilin composition is described in EP 437,621 A1. Aqueous solutions of motilin and freeze-dried compositions showed stability in a solution at a pH of 4.0-5.5. The motilins described therein included canine motilin, swine motilin, human motilin, and certain motilin derivatives (e.g., ¹³leucine-swine motilin). These motilins and motilin derivatives retained approximately 94% motilin content after 7 days storage at 60° C.

While the prior art recognizes the stabilizing effect of certain solutions on selected peptides, and EP 437,621 A1 suggests that native (full length) motilin can be prepared as stable aqueous or freeze-dried compositions, the results provided therein are not predictive of how other motilin derivatives or motilin-like peptides would behave in similar aqueous solutions or lyophilized compositions. That is because certain differences in peptide structure and the existence or absence of charged and acid/base functional groups for a given peptide can significantly affect the solubility, pH/solubility profile, isoelectric point, and pKa values, thus making one peptide sufficiently different from other related peptides. Accordingly, one cannot predict with certainty whether a stable aqueous or lyophilized formulation of a motilin derivative can be prepared.

As described in U.S. Pat. No. 5,422,341, the motilin-like peptides are structurally distinct from native motilin. These structural differences, which are described in more detail below, make determining whether the motilin-like peptide would retain its stability in an aqueous or lyophilized solution difficult.

Because of the above-described advantages provided by the motilin-like peptides, it would be desirable to provide aqueous pharmaceutical compositions including such motilin-like peptides. It would also be desirable to provide compositions including such motilin-like peptides that can be administered to a patient after long-term storage. In addition, it would be desirable to provide compositions that substantially prevent degradation of the motilin-like peptide, but where maximum levels of potency of the compound during the storage period are maintained.

It would also be desirable to provide a ready-to-use aqueous peptide composition that remains stable (and maintains potency) after storage at ambient temperature conditions, as well as after exposure to elevated temperatures, such as, but not limited to the temperature of steam sterilization. It would be desirable to provide such synthetic motilin-like peptides as aqueous compositions in varying concentrations, or in a lyophilized form. It would be desirable to provide a composition including a synthetic motilin-like peptide that, if necessary, can be stored in and remain stable for extended periods of time in any one of several different containers. Finally, it would be desirable to provide a motilin-like peptide in an aqueous environment without the need for a separate buffer.

### SUMMARY OF THE INVENTION

In one aspect, the present invention is directed to pharmaceutical compositions comprising approximately 0.5 μg/ml to 100 mg/ml of a synthetic motilin-like peptide having no more than 16 amino acids and structure described below.

The composition may be provided as aqueous compositions having a pH of between 3 and 9 and an osmolality of approximately 10-500 mOsm/kg.

In still a further aspect, the present invention is directed to compositions including a synthetic motilin-like peptide having the structure described below and including no more than 16 amino acids, wherein the composition is provided in a concentrated form including a selected amount of peptide whereby upon dilution the aqueous composition includes 0.5 μg/ml-100 μg/ml of the peptide, having a pH of between 3-9 and an osmolality of approximately 10-500 mOsm/kg.

In still a further aspect, the present invention is directed to lyophilized compositions including a synthetic motilin-like peptide having the structure as described below and including no more than 16 amino acids whereby upon reconstitution, the reconstituted composition includes 0.5 μg/ml-100 μg/ml of the peptide and has a pH of between 3-9 and an osmolality of approximately 10-500 mOsm/kg.

### BRIEF DESCRIPTION OF THE DRAWINGS

FIG. 1 is a graph showing the stability of the peptide in a buffered composition embodying the present invention up to 24 months of storage.

FIG. 2 is a graph showing the stability of the peptide in a composition embodying the present invention after autoclaving.

FIG. 3 is a graph showing the stability profile of the synthetic motilin-like peptide at a concentration of 5 μg/ml in a buffered composition embodying the present invention when it is stored in a selected glass container.

FIG. 4 is a graph showing the stability profile of the synthetic motilin-like peptide at a concentration of 30 μg/ml in a buffered composition embodying the present invention when it is stored in a selected glass container.

FIG. 5 is a graph showing the stability of the peptide in a composition embodying the present invention after storage, wherein the composition is lyophilized, stored in its lyophilized form, and subsequently reconstituted.

FIG. 6 is a graph showing the stability of the peptide in an unbuffered aqueous composition embodying the present invention after 14 days of storage.

FIG. 7 is a graph showing the stability of the peptide in an unbuffered composition embodying the present invention after autoclaving and storage for up to 14 days.

### DETAILED DESCRIPTION

Pharmaceutical compositions of the present invention include a synthetic motilin-like peptide that may be effective in the treatment of gastrointestinal disorders such as, but not limited to, post-operative ileus, diabetic gastroparesis and paralytic ileus. The pharmaceutical compositions of the present invention include a synthetic motilin-like peptide, or a pharmaceutically acceptable salt thereof, in a selected buffer at a selected concentration. The pharmaceutical compositions have a pH and osmolality also selected to maintain stability of the peptide over an extended period of time.

The pharmaceutical compositions of the present invention remain stable during long-term storage, such as at least 12 months, and preferably at least 18 months and up to 24 months, at temperatures commonly encountered during storage, such as, but not limited to, between approximately 4° C. and approximately 30° C. The pharmaceutical compositions of the present invention show minimal degradation and loss of the peptide potency over such periods of time and at such temperatures. The motilin-like peptide of the pharmaceutical compositions of the present invention remain chemically stable and biologically potent even after exposure to elevated temperatures, such as, but not limited to, the temperature of steam sterilization (e.g., 121° C.). As used herein, references to stability refer to the degree of peptide degradation and residual potency of the peptide as determined by biological assays.

For example, a motilin-like peptide that is 95% undegraded after storage, as determined by chemical purity analysis, retains 95% potency in biological assays. In the pharmaceutical compositions of the present invention, the motilin-like peptide does not substantially degrade and remains substantially potent and effective in the treatment of gastrointestinal disorders.

Pharmaceutical compositions of the present invention include, in general, a selected amount of a synthetic motilin-like polypeptide. The synthetic peptide may be a motilin-like peptide that is effective in treating or stimulating gastrointestinal motor activity.

Motilin-like peptides for stimulating gastrointestinal activity show a high affinity for the motilin receptor. Examples of such motilin-like polypeptides are described in U.S. Pat. No. 5,422,341, previously incorporated by reference, which discloses amino acid sequences of synthetic motilin-like peptides.

In a preferred embodiment of the present invention, the synthetic motilin-like peptide contained in the compositions of the present invention is truncated. Preferably, the synthetic motilin-like peptide will typically include no more than 16 amino acids and, more preferably, 11-16 amino acids.

In one embodiment, the motilin-like peptide has the following structure: 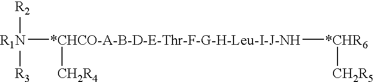
where:
A is the L-stereoisomer of a lipophilic aliphatic amino acid;
B is L-proline or L-alanine;
D is the L-stereoisomer of a lipophilic aliphatic amino acid;
E is the L-stereoisomer of an aromatic, lipophilic aliphatic, or alicyclic amino acid;
F is the L-stereoisomer of an aromatic or heteroaromatic amino acid;
G is glycine or D-alanine;
H is L-glutamic acid or L-glutamine;
I is L-glutamine;
J is selected from the group consisting of Z, Z-Leu, Z-Leu-Gln, Z-Leu-Gln-Glu, Z-Leu-Gln-Glu-Lys, wherein Z is selected from the group consisting of D-arginine, D-homoarginine, D-glutamine, D-asparagine, and D-alanine;
R₁ is lower-alkyl or allyl;
R₂ is selected from the group consisting of hydrogen, lower-alkyl, propargyl, and allyl;
R₃ is selected from the group consisting of hydrogen, lower-alkyl, and allyl;
R₄ is cycloalkyl or aryl which may be unsubstituted or substituted with one or more substituents selected from the group consisting of halogen, hydroxy, and lower-alkoxy;
R₅ is selected from the group consisting of —CH₂ CONH₂, aminoalkyl groups containing from 1 to 3 carbon atoms, and guanidinoalkyl groups containing 2 or 3 carbon atoms;
R₆ is —COOH or —CONH₂; and the symbol * represents an asymmetric carbon atom which may be in the D or L configuration, and each lower-alkyl group contains from 1 to 4 carbon atoms, with the proviso that:
(i) R₅ is —CH₂ CONH₂ only when J is Z-Leu;

In one preferred embodiment, the peptide contained in the pharmaceutical composition of the present invention may further include D-Arginine at position 12. The peptide of the present invention may also include an alkylated and, preferably, tri-alkylated (for example, methylated) N-terminus. Additionally, the synthetic motilin-like peptide may include an amide (—CONH₂) group at the C terminal carboxylic acid functionality of the polypeptide chain, which changes the pKa profile and charge of the motilin-like peptide (as compared to native motilin) and is also believed to affect solubility.

A particularly preferred embodiment of the present invention includes the synthetic motilin-like peptide receptor agonist including SEQ ID NO.: 1 and represented by the following formula: (Me₃N)⁺Phe-Val-Pro-Ile-Phe-Thr-Tyr-Gly-Glu-Leu-Gln-D-Arg-Leu-Lys-NH₂, where the following abbreviations represent the amino acids:

Phe—L-Phenylalanine

Tyr—L-Tyrosine

Leu—L-Leucine

Val—L-Valine

Pro—L-Proline

Ile—L-Isoleucine

Thr—L-Threonine

Gly—Glycine

Glu—L-Glutamic acid

Gln—L-Glutamine

D-Arg—D-Arginine

Lys.—L-Lysine.

Motilin-like peptides, including SEQ ID NO.: 1, are particularly effective and display enhanced stability, extended potency and suitability to sterilization by autoclaving (described below), when such peptides are included in the compositions of the present invention. Also included within the scope of the present invention are all pharmaceutically acceptable salts of the above-identified peptide.

Other peptides, including those disclosed in U.S. Pat. No. 5,422,341, that are substantially similar to the peptide including SEQ ID NO.: 1 described herein, may also display enhanced stability in the compositions of the present invention. This may include, for example, other synthetic motilin-like peptides described in the referenced patents which have substantial homology (i.e., greater than 50% and preferably at least 60%-70%) with the N-terminal sequence of the human motilin peptide.

The motilin-like peptide contained in the compositions of the present invention is provided in an amount between approximately 0.5 μg/ml to 100 mg/ml. More preferably, where the composition is “ready-to-use,” (i.e., not requiring further dilution or reconstitution) the amount of motilin-like peptide may be between approximately 1 μg/ml and 1 mg/ml, even more preferably 1 μg/ml-50 μg/ml and typically in the range of 1 μg/ml-30 μg/ml.

Because of the potency of the peptides, compositions of low concentration may be advantageous in the controlled administration of the desired amount of the peptides. Of course, a more concentrated composition of the peptide, still embodying the present invention, may be diluted to produce a solution for convenient administration.

In one embodiment, where the composition is provided in a “ready-to-use” form, the motilin-like peptide may be dissolved in a buffer solution to provide an aqueous composition. The buffer is selected to maintain the pH of the pharmaceutical composition between approximately 3 and 9, at which pH the peptide shows substantially enhanced stability and retention of potency. More preferably, the pH of the pharmaceutical compositions is between approximately 4 and 6, more preferably 4.5 and 5.5 and, most preferably, between approximately 4.8-5.2. At these pH levels, the peptide of SEQ. ID. 1, a preferred example of a motilin-like peptide, shows only minimal degradation during storage. (The preferred pH of the composition may also be determined, in part, based on whether the composition is prepared as an aqueous composition or a lyophilized formulation, discussed below.)

Buffer solutions suitable for use with the pharmaceutical compositions of the present invention include buffers such as sodium acetate/acetic acid, sodium hydrogen phosphate buffers and sodium citrate/citric acid. Other buffer systems known to those of skill in the art, such as, but not limited to, tartarate, succinate, TRIS, histidine and glycine, may also provide adequate buffering of the compositions within the above-identified pH range.

As indicated above, in one embodiment, sodium acetate/acetic acid buffer is combined with the motilin-like peptide to provide an aqueous composition. When provided as a ready-to-use solution, the concentration of the buffer in the pharmaceutical composition may be between 5 and 250 mM and, more preferably, between 5 and 50 mM, with a concentration of approximately 5-25 mM being most preferred for many of the embodiments, to achieve and maintain the preferred pH.

Where the buffered composition is provided as a concentrated solution requiring dilution, the concentration of buffer may be significantly higher, such as, but not limited to, up to 10 times higher or up to 2-3 M. (In such concentrated solutions, the concentration of peptide will also be correspondingly higher.) The concentrated compositions are then diluted with a diluent to arrive at the preferred concentration and amounts of buffer (e.g. 5-250 mM) and peptides (e.g., 1 μg/ml-100 μg/ml) at a tonicity suitable for administration. Suitable, pharmaceutically acceptable diluents include, but are not limited to sterile water, sterile sodium chloride solution, sterile solutions of dextrose (e.g., 5%) and other sugars, and the like.

The aqueous pharmaceutical compositions of the present invention may be either hypotonic, substantially isotonic or hypertonic, having an osmolality of between 10-500 mOsm. More preferably, however, the composition is isotonic, or mildly hypotonic or mildly hypertonic, although hypotonic compositions with an osmolality under 100 mOsm and even under 50 mOsm may also be effective. Typically, however, the tonicity of the pharmaceutical composition may be between 270-320 mOsm. The tonicity of the pharmaceutical composition may be adjusted using sodium chloride (NaCl) or other tonicity adjusting additives such as glycerine, mannitol, sucrose and other reduced sugars, or other agents known to those skilled in the art, as necessary.

Table I, below, summarizes the preferred constituents and conditions of the buffered pharmaceutical compositions of the present invention in both ready-to-use and concentrated forms.

TABLE I

| Component/Property | Amount |
|---|---|
| Peptide (μg/ml) | 0.5-100,000 |
| Buffer (mM) | 5-2500 |
| pH | 3-9 |
| Osmolality (mOsm/kg) | 10-1000 |

In a preferred ready-to-use, buffered, embodiment, aqueous pharmaceutical compositions of the present invention include 1 μg/ml-100 μg/ml of the synthetic, motilin-like peptide of SEQ. ID. No. 1 in approximately 10 mM sodium acetate/acetic acid buffer at a pH of approximately 4.8-5.2 and an osmolarity of approximately 300±20 mOsm.

In addition to aqueous, ready-to-use compositions, or concentrated compositions requiring dilution, the composition of the present invention may also be stored in a lyophilized (freeze-dried) form. The composition, in its lyophilized form, may include the relative amounts of peptide and buffer set forth in Table I above (as well as other agents). It will be appreciated that where the composition has been stored in a lyophilized form, administration of the composition may occur immediately upon reconstitution, making long-term storage stability of the reconstituted composition less critical and, thus, the presence of a buffer, optional. In any event, in one embodiment of the lyophilized formulation, the optional buffer may be citrate/citric acid at a preferred pH of approximately 4.7 and more preferably 6.0. In addition, the lyophilized formulation may further include one or more bulking agents such as, but not limited to, sucrose, mannitol and glycine and/or combinations thereof. Bulking agents may be included in a concentration of 0.5-15% w/v.

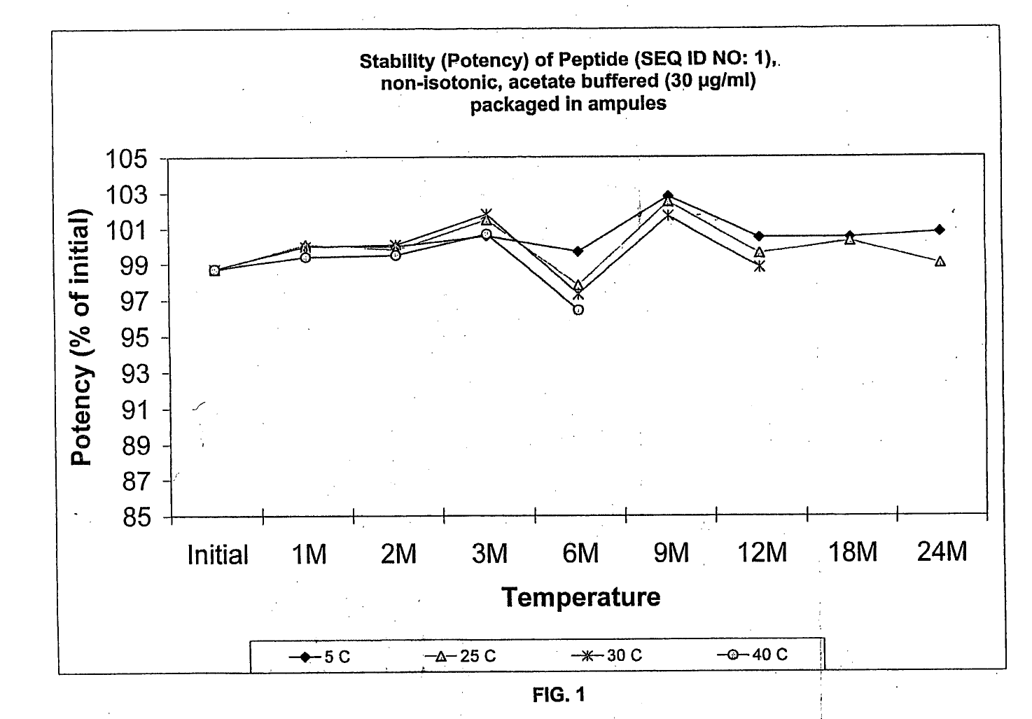

The aqueous pharmaceutical compositions of the present invention provides benefits previously unknown. The motilin-like peptide of pharmaceutical compositions of the present invention remains stable when stored over a wide range of temperatures for extended periods of time. For example, when stored at room temperature or its preferred lower storage temperature of 4° C.-5° C., substantial stability is maintained for significant periods of time. For example, at a storage temperature of approximately 25° C., the peptide contained in the composition of the present invention remains stable for at least 12 months, and more preferably at least approximately 18 months and, even more preferably, up to and even beyond approximately 24 months, retaining a potency of 90% or more as shown, for example, in FIG. 1. At a storage temperature of 5° C., storage with only marginal degradation (and a potency of at least 90-95%) of up to at least 18 months is regularly observed.

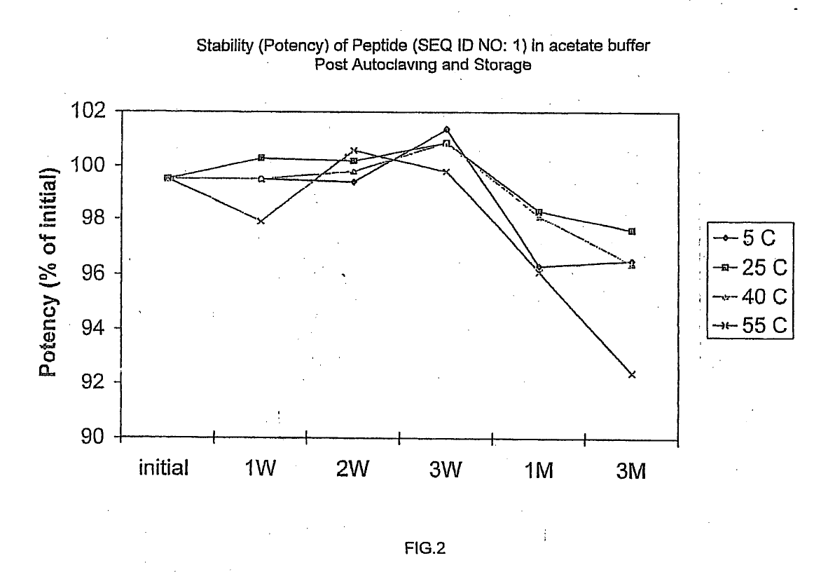

A further advantage of the pharmaceutical composition of the present invention is its peptide stability when the composition is subjected to elevated temperatures such as the temperature of steam sterilization. Many peptides are inherently unstable and, when subjected to heat, are known to degrade very rapidly, typically in a few minutes or less. In contrast, in compositions of the present invention, the peptide substantially retains its stability (i.e., remains substantially undegraded and retains a substantial percentage of its initial potency), even after the composition is (1) subjected to steam sterilization (for at least 10 minutes and up to 60 minutes at temperatures of approximately 100° C.-128° C. and typically 121° C.), and (2) subsequently stored in a suitable container (described below) at lower temperatures typically encountered by a pharmaceutical composition, such as about 4° C.-5° C. to about 25° C. As shown in FIG. 2 and described in Example 1 below, in samples of a buffered pharmaceutical composition of the present invention that were subjected to steam sterilization in a Type I glass vial container, and subsequently stored in such container, the peptide retained a significant percentage of its initial potency (and binding affinity) after 3 months of storage at 5° C., 25° C., 40° C., and even at 55° C.

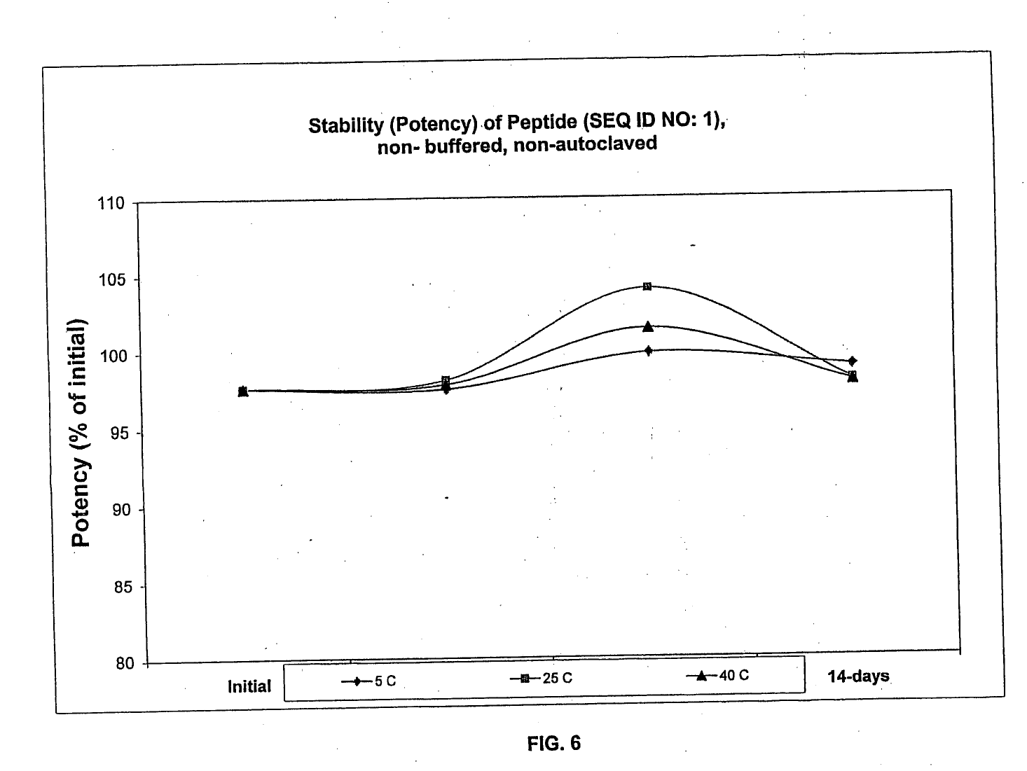

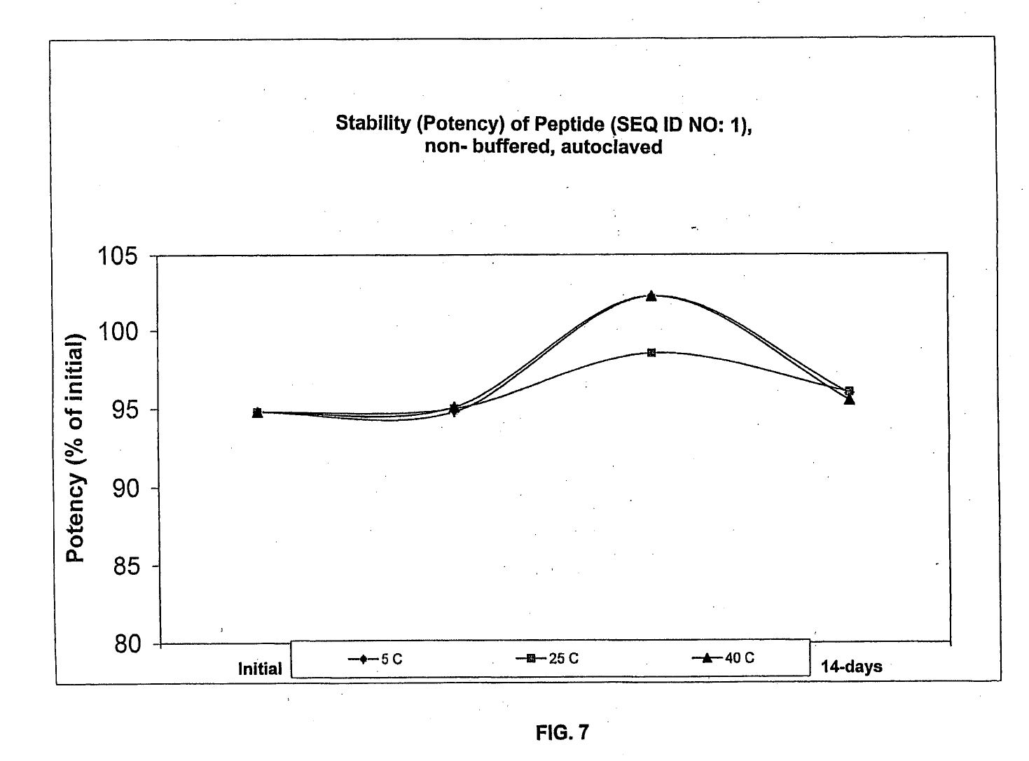

In another embodiment, the composition may be provided as an aqueous composition with the peptide previously described (i.e., no more than 16 amino acids), but without the inclusion of a buffer. The compositions may have a pH of between 3 and 9 and an osmolality of approximately 10-100 mOsm/kg. Aqueous compositions of the present invention, without buffer, remain stable for at least 14 days when stored at different temperatures as shown, for example, in FIGS. 6 and 7. As further shown in FIGS. 6 and 7, such compositions retain peptide stability even after autoclaving for 30 minutes at approximately 121° C.

Preventing degradation of the peptide and, thereby, retaining potency and binding affinity can be further enhanced by storing the compositions of the present invention in a suitable container. While many different types of glass and some plastics have proved satisfactory, for longer term storage, vapor-treated silicon vials are preferred. Such Type 1 Plus vapor-treated silicon vials are available, for example, from Schott of Lebanon, Pa., under the product name Schott Type 1 Plus silica coated vials. Other suitable storage containers include Type 1 USP vials, Type I glass ampule and pre-packed glass syringes.

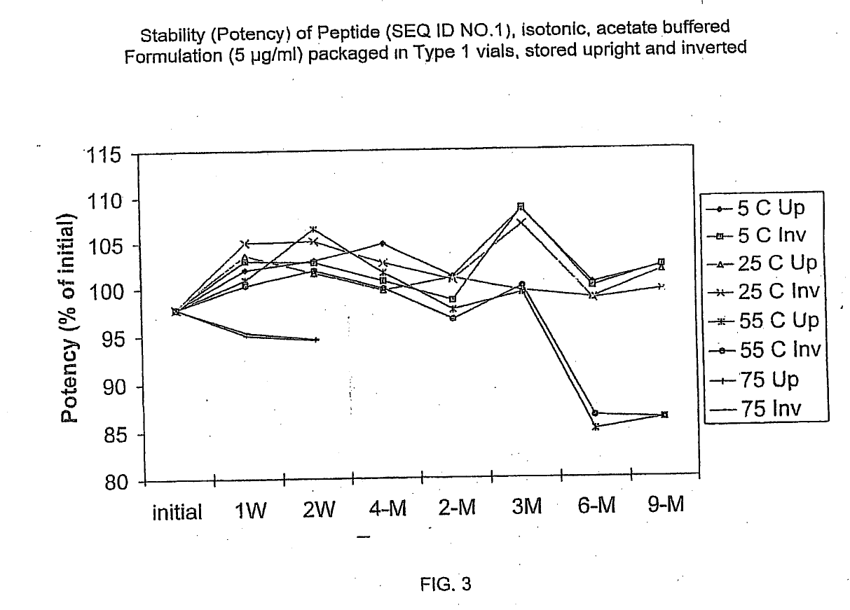

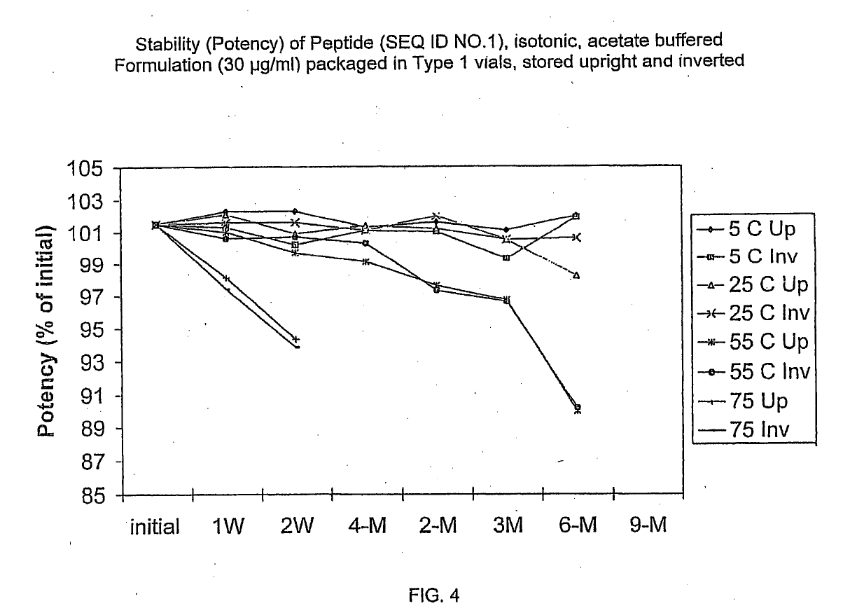

Some plastics have also proven to be effective in assisting in the stabilization of the peptide composition. In this regard, preferred plastics are cyclic olefin copolymers, available, for example, from West Pharmaceuticals under the product name CZ resin vials, or from Schott under the product name TopPac vials, or from Alcan packaging under the product name Ticona Topas serum vials. The compositions of the present invention may also be stored in PVC, as well as non-PVC flexible bags. When stored in the above described glass or plastic containers, the above-described storage times at the above-described storage temperatures can be achieved, although for storage periods of at least 18 months, Type I Plus (vapor treated silicon) vials and Type I Plus glass ampules are preferred. As shown, for example in FIGS. 3 and 4, storage of an aqueous buffered composition of the present invention which included approximately 5 μg/ml (FIG. 3) or 30 μg/ml (FIG. 4) of the synthetic motilin-like peptide of sequence SEQ ID 1 in Schott Type 1 vials demonstrated stability (and maintenance of high levels of potency) for several months when stored in at temperatures of 5° C. to 55° C. It will be appreciated by those skilled in the art that the measured stability of pharmaceutical compositions at elevated temperatures, such as 40° C. or higher, may serve as the basis of prediction of stability for longer periods at more typical storage temperatures, such as 20°-25° C. or 4°-8° C., for example. Such “accelerated storage” studies are common in the field of the present invention.

### EXAMPLE 1

### Stability of Motilin-Like Peptide following Autoclaving and Storage

The motilin-like peptide of SEQ ID NO.: 1 was dissolved at a concentration of 30 μg/ml in 10 mM sodium acetate buffer, pH 5.0. Samples of the peptide solution stored in Type I glass vials were autoclaved at 121° C. for 15 minutes and the autoclaved samples were incubated at 5° C., 25° C., 40° C., or 55° C. The potency of the peptide following storage at each temperature was measured over time by testing samples after incubation of one week, two weeks, three weeks, one month and three months. Potency was determined by using HPLC to measure the amount of undegraded peptide remaining in each sample.

As shown in FIG. 2, the peptide retained greater than 90% potency after autoclaving and storage for 3 months even at an elevated storage temperature of 55° C. The pharmaceutical composition of the present invention shows remarkable stability over the storage period even at elevated storage temperature. It will be appreciated by those skilled in the art that stability data obtained at such elevated temperatures (i.e., accelerated storage conditions) may be used to predict stability at lower, more typical storage temperatures over extended periods of time.

### EXAMPLE 2

### Stability of Lyophilized Formulations of Motilin-Like Peptide Compositions

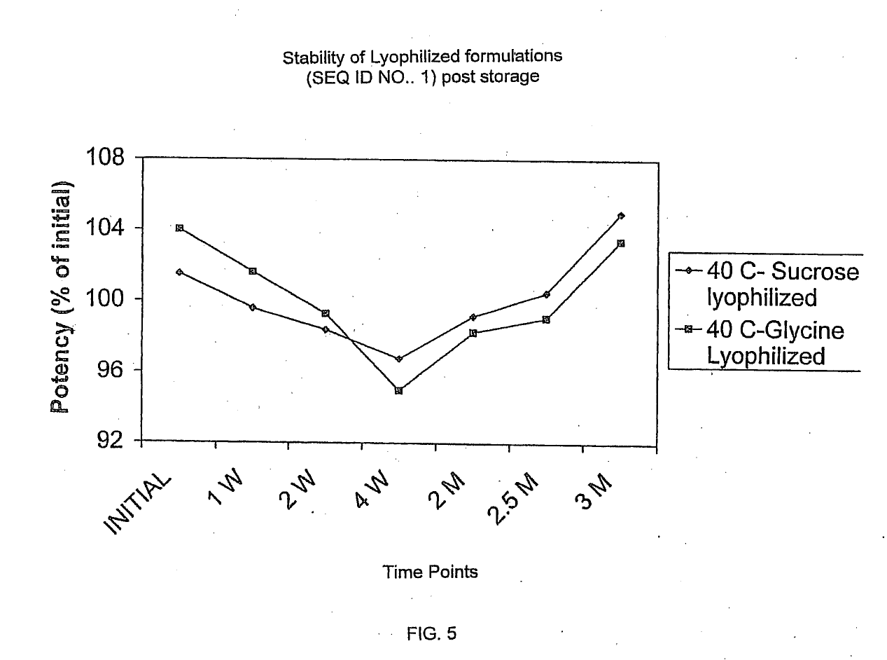

The motilin-like peptide including SEQ ID NO.: 1 was dissolved at a concentration of 30 μg/ml in 10 mM citrate buffer and 1% w/v of sucrose (bulking agent) at pH 6.0. The composition was lyophilized by standard techniques and stored in a 20 ml vial at 40° C. The potency of the motilin-like peptide was measured at the time intervals indicated in FIG. 5, by reconstituting the composition in water. Potency was determined by using HPLC to measure the amount of undegraded peptide remaining in each sample. As shown in FIG. 5, the lyophilized motilin-like peptide retained a significant level of potency (i.e., greater than 90%) for several months. Similar results were obtained when the sucrose bulking agent was replaced with a combined 0.04% w/v glycine and 0.4% w/v mannitol bulking agent. (Again, it will be appreciated by those skilled in the art that the stability of pharmaceutical compositions at elevated temperatures, such as 40° C. as in FIG. 5, may serve as the basis of prediction of stability for longer periods at more typical storage temperatures, such as approximately 20° C.-25° C. or 4° C.-8° C., for example).

Whether prepared and stored as an aqueous unbuffered solution, an aqueous buffered solution, a ready-to-use or concentrated, or a lyophilized composition that is subsequently reconstituted, the pharmaceutical compositions of the present invention remains potent during storage for extended periods of time. The maintained peptide potency of around 90-100%, and in some instances, greater than 95% during storage at 5, 25, 40 and 55° C. for at least 18 months, makes the pharmaceutical composition of the present invention a preferred means for the therapeutic delivery of motilin-like peptides.

While the present invention has been described in the context of its preferred embodiments, numerous modifications to such embodiments are possible without departing from the spirit of the present invention, which is set forth in the accompanying claims.

That which is claimed:

1. An aqueous pharmaceutical composition comprising:
(a) approximately 0.5 μg/ml to 100 mg/ml of a synthetic motilin-like peptide having no more than 16 amino acids and having the following structure: 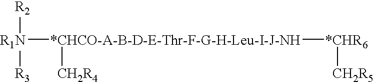
where:
A is the L-stereoisomer of a lipophilic aliphatic amino acid;
B is L-proline or L-alanine;
D is the L-stereoisomer of a lipophilic aliphatic amino acid;
E is the L-stereoisomer of an aromatic, lipophilic aliphatic, or alicyclic amino acid;
F is the L-stereoisomer of an aromatic or heteroaromatic amino acid;
G is glycine or D-alanine;
H is L-glutamic acid or L-glutamine;
I is L-glutamine or L-alanine;
J is selected from the group consisting of Z, Z-Leu, Z-Leu-Gln, Z-Leu-Gln-Glu, Z-Leu-Gln-Glu-Lys, wherein Z is selected from the group consisting of D-arginine, D-homoarginine, D-glutamine, D-asparagine, and D-alanine;
R₁ is lower-alkyl or allyl;
R₂ is selected from the group consisting of hydrogen, lower-alkyl, propargyl, and allyl;
R₃ is selected from the group consisting of hydrogen, lower-alkyl, and allyl;
R₄ is cycloalkyl or aryl which may be unsubstituted or substituted with one or more substituents selected from the group consisting of halogen, hydroxy, and lower-alkoxy;
R₅ is selected from the group consisting of —CH₂ CONH₂, aminoalkyl groups containing from 1 to 3 carbon atoms, and guanidinoalkyl groups containing 2 or 3 carbon atoms;
R₆ is —COOH or —CONH₂; and
the symbol * represents an asymmetric carbon atom which may be in the D or L configuration, and each lower-alkyl group contains from 1 to 4 carbon atoms, with the proviso that:
(i) R₅ is —CH2 CONH2 only when J is Z-Leu or Z-Leu-Gln-Glu-Lys-Glu-Arg-Asn-Lys-Gly;
said composition having a pH of between 3 and 9 and having an osmolality of approximately 10-500 mOsm/kg;

2. The composition of claim 1 wherein said synthetic motilin-like peptide has 14 amino acids.

3. The composition of claim 1 wherein said synthetic motilin-like peptide is (Me₃N)⁺Phe-Val-Pro-Ile-Phe-Thr-Tyr-Gly-Glu-Leu-Gln-D-Arg-Leu-Lys-NH₂.

4. The composition of claim 1 wherein the amino acid at position 12 of said motilin-like peptide is D-Arginine.

5. A concentrated aqueous pharmaceutical composition including:
a) a selected amount of a synthetic motilin-like peptide having no more than 16 amino acids and having the structure: 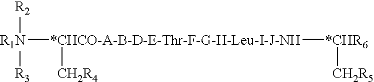
where:
A is the L-stereoisomer of a lipophilic aliphatic amino acid;
B is L-proline or L-alanine;
D is the L-stereoisomer of a lipophilic aliphatic amino acid;
E is the L-stereoisomer of an aromatic, lipophilic aliphatic, or alicyclic amino acid;
F is the L-stereoisomer of an aromatic or heteroaromatic amino acid;
G is glycine or D-alanine;
H is L-glutamic acid or L-glutamine;
I is L-glutamine or L-alanine;
J is selected from the group consisting of Z, Z-Leu, Z-Leu-Gln, Z-Leu-Gln-Glu, Z-Leu-Gln-Glu-Lys, wherein Z is selected from the group consisting of D-arginine, D-homoarginine, D-glutamine, D-asparagine, and D-alanine;
R₁ is lower-alkyl or allyl;
R₂ is selected from the group consisting of hydrogen, lower-alkyl, propargyl, and allyl;
R₃ is selected from the group consisting of hydrogen, lower-alkyl, and allyl;
R₄ is cycloalkyl or aryl which may be unsubstituted or substituted with one or more substituents selected from the group consisting of halogen, hydroxy, and lower-alkoxy;
R₅ is selected from the group consisting of —CH₂ CONH₂, aminoalkyl groups containing from 1 to 3 carbon atoms, and guanidinoalkyl groups containing 2 or 3 carbon atoms;
R₆ is —COOH or —CONH₂; and
the symbol * represents an asymmetric carbon atom which may be in the D or L configuration, and each lower-alkyl group contains from 1 to 4 carbon atoms, with the proviso that:
(i) R₅ is —CH2 CONH2 only when J is Z-Leu or Z-Leu-Gln-Glu-Lys-Glu-Arg-Asn-Lys-Gly;
and
b) a selected amount of a buffer,
whereby upon dilution said diluted aqueous pharmaceutical composition comprises:
i) 0.5 μg/ml to 100 μg/ml of said peptide;
ii) having a pH of between 3-9 and an osmolality of approximately 10-500 mOsm/kg.

6. A lyophilized pharmaceutical composition including:
a) a synthetic motilin-like peptide having no more than 16 amino acids having the structure: 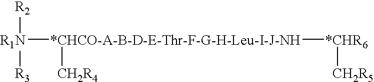
where:
A is the L-stereoisomer of a lipophilic aliphatic amino acid;
B is L-proline or L-alanine;
D is the L-stereoisomer of a lipophilic aliphatic amino acid;
E is the L-stereoisomer of an aromatic, lipophilic aliphatic, or alicyclic amino acid;
F is the L-stereoisomer of an aromatic or heteroaromatic amino acid;
G is glycine or D-alanine;
H is L-glutamic acid or L-glutamine;
I is L-glutamine or L-alanine;
J is selected from the group consisting of Z, Z-Leu, Z-Leu-Gln, Z-Leu-Gln-Glu, Z-Leu-Gln-Glu-Lys, wherein Z is selected from the group consisting of D-arginine, D-homoarginine, D-glutamine, D-asparagine, and D-alanine;
R₁ is lower-alkyl or allyl;
R₂ is selected from the group consisting of hydrogen, lower-alkyl, propargyl, and allyl;
R₃ is selected from the group consisting of hydrogen, lower-alkyl, and allyl;
R₄ is cycloalkyl or aryl which may be unsubstituted or substituted with one or more substituents selected from the group consisting of halogen, hydroxy, and lower-alkoxy;
R₅ is selected from the group consisting of —CH₂ CONH₂, aminoalkyl groups containing from 1 to 3 carbon atoms, and guanidinoalkyl groups containing 2 or 3 carbon atoms;
R₆ is —COOH or —CONH₂; and
the symbol * represents an asymmetric carbon atom which may be in the D or L configuration, and each lower-alkyl group contains from 1 to 4 carbon atoms, with the proviso that:
(i) R₅ is —CH2 CONH2 only when J is Z-Leu or Z-Leu-Gln-Glu-Lys-Glu-Arg-Asn-Lys-Gly;
whereby upon reconstitution, said reconstituted aqueous composition comprises:
i) 0.5 μg/ml to 100 μg/ml of said peptide;
ii) having a pH of between 3-9 and an osmolality of approximately 10-500 mOsm/kg.
